(19) United States
(12) Patent Application Publication
(10) Pub. No.: US 2015/0217037 A1
(43) Pub. Date: Aug. 6, 2015

(54) Fluid Isolator for Breast Pump Systems

(71) Applicants: Ashia M. Pollen, Madison, WI (US); Robert J. Harter, La Crosse, WI (US)

(72) Inventors: Ashia M. Pollen, Madison, WI (US); Robert J. Harter, La Crosse, WI (US)

(21) Appl. No.: 14/685,689

(22) Filed: Apr. 14, 2015

(51) Int. Cl. A61M 1/06

(52) U.S. Cl. CPC A61M 1/06

(57) ABSTRACT

A breast pump system includes a fluid isolator that is particularly useful as an aftermarket product that can be readily installed between various vacuum pumps and milk collection devices. The fluid isolator includes a pliable, limp diaphragm that equalizes the pressure between a vacuum pump and a milk collection device while providing a barrier that prevents milk from accidentally backflowing from the milk collection device to the vacuum pump. In some examples, the fluid isolator includes one or more tiny supplementary openings for synchronizing the movement of the diaphragm with the cyclical action of the vacuum pump and/or for returning a misdirected milk droplet in a suction tube back to a charging chamber of the milk collection device.

### FIELD OF THE DISCLOSURE

The subject invention generally pertains to human breast milk collection systems and more specifically to means for inhibiting milk from backflowing to a vacuum pump.

### BACKGROUND

Breast pump systems are used for collecting breast milk expressed from a lactating woman. Some breast pump systems have a milk collection device with a funnel that fittingly receives the woman's breast. In many cases, a vacuum pump provides cyclical periods of positive and negative pressure to the milk collection device. During periods of negative pressure (subatmospheric pressure), vacuum delivered to the device withdraws a small discrete volume of milk from the breast and conveys that charge of milk to a small charging chamber. During each period of positive pressure, lightly pressurized air relaxes the breast momentarily and at the same time forces the charge of milk from the charging chamber to a larger milk storage chamber. The cycle repeats until the storage chamber is full or the woman is finished “pumping.”

Some breast pump systems have a milk collection device that is worn within the cup of a common brassiere. Examples of such systems are disclosed in U.S. Pat. Nos. 7,559,915; 8,118,772; and 8,702,646; all of which are incorporated herein by reference. Other breast pump systems have funnels that are handheld or are supported by or extend through a special purpose brassier. Examples of such systems are disclosed in U.S. Pat. Nos. 5,941,847; 7,094,217; and 8,057,452; all of which are incorporated herein by reference.

### BRIEF DESCRIPTION OF THE DRAWINGS

FIG. 1 is a cross-sectional side view of an example milk collection device constructed in accordance with the teachings disclosed herein.

FIG. 2 is a combination schematic diagram and cross-sectional side view similar to FIG. 1 but showing the milk collection device as part of an example breast pump system.

FIG. 3 is a view similar to FIG. 2 but showing the system during a positive pressure period rather than a suction pressure period.

FIG. 4 is a cross-sectional side view of the milk collection device shown in FIGS. 1-3, but showing the device fully tipped over and pointed down.

FIG. 5 is a cross-sectional view of the milk collection device shown in FIG. 1 but showing the device in a disassembled cleaning state.

FIG. 6 is a cross-sectional view similar to FIG. 1 but with the outer shell omitted.

FIG. 7 is a cross-sectional view showing a portion of FIG. 6.

FIG. 8 is a cross-sectional view taken along line 8-8 of FIG. 7.

FIG. 9 is a cross-sectional view showing a portion of FIG. 6.

FIG. 10 is a cross-sectional view taken along line 10-10 of FIG. 9.

FIG. 11 is a cross-sectional view showing a portion of FIG. 6.

FIG. 12 is a cross-sectional view taken along line 12-12 of FIG. 11.

FIG. 13 is a cross-sectional view showing a portion of FIG. 6.

FIG. 14 is a cross-sectional view taken along line 14-14 of FIG. 13.

FIG. 15 is a cross-sectional view similar to FIG. 10 but showing an airflow pattern during a negative pressure period (first period).

FIG. 16 is a cross-sectional view similar to FIG. 15 but showing an airflow pattern during a positive pressure period (second period).

FIGS. 17 and 18 are illustrations demonstrating an example “vacuum break” concept.

FIG. 19 is an illustration demonstrating another example “vacuum break” concept.

FIG. 20 is a cross-sectional view similar to FIG. 1 but showing another example milk collection device constructed in accordance with the teachings disclosed herein.

FIG. 21 is a cross-sectional view similar to FIG. 1 but showing another example milk collection device constructed in accordance with the teachings disclosed herein.

FIG. 22 is a cross-sectional view similar to FIG. 1 but showing of another example milk collection device constructed in accordance with the teachings disclosed herein.

FIG. 23 is a combination schematic diagram and cross-sectional side view of an example fluid isolator being added to an example breast pump system, both of which are constructed in accordance with the teachings disclosed herein.

FIG. 24 is an exploded view of the example fluid isolator shown in FIG. 23.

FIG. 25 is a combination schematic diagram and cross-sectional view similar to FIG. 23 but showing the breast pump system after the installation of the fluid isolator.

FIG. 26 is a cross-sectional view taken along line 26-26 of FIG. 25.

FIG. 27 is a combination schematic diagram and cross-sectional view similar to FIGS. 2 and 25 with the vacuum pump in a negative pressure state.

FIG. 28 is a combination schematic diagram and cross-sectional view similar to FIGS. 3 and 25 with the vacuum pump in a positive pressure state.

FIG. 29 is a combination schematic diagram and cross-sectional side view of another example breast pump system constructed in accordance with the teachings disclosed herein.

### DETAILED DESCRIPTION

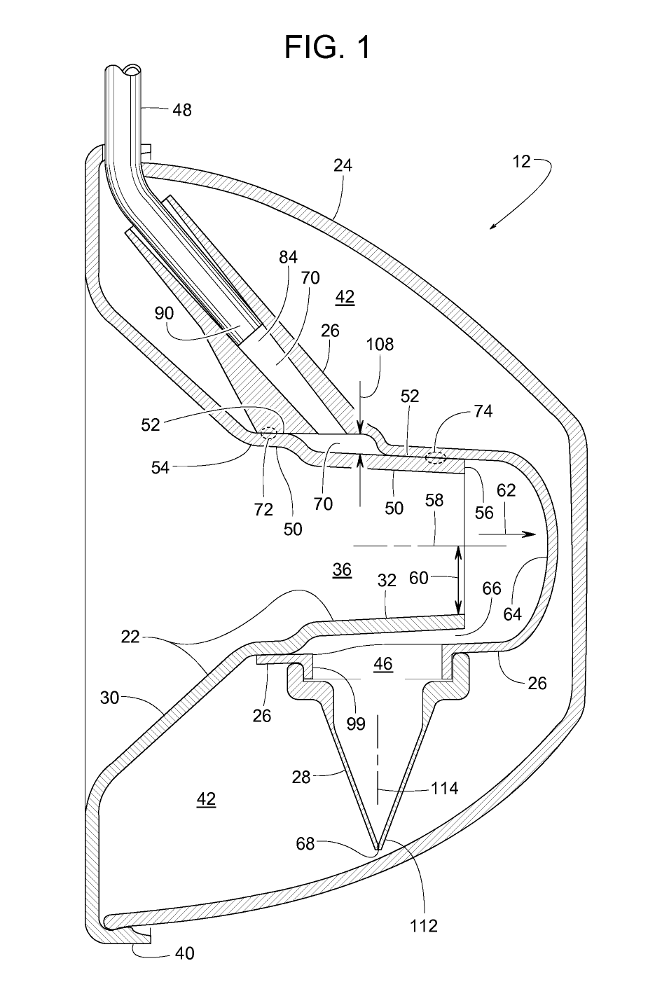

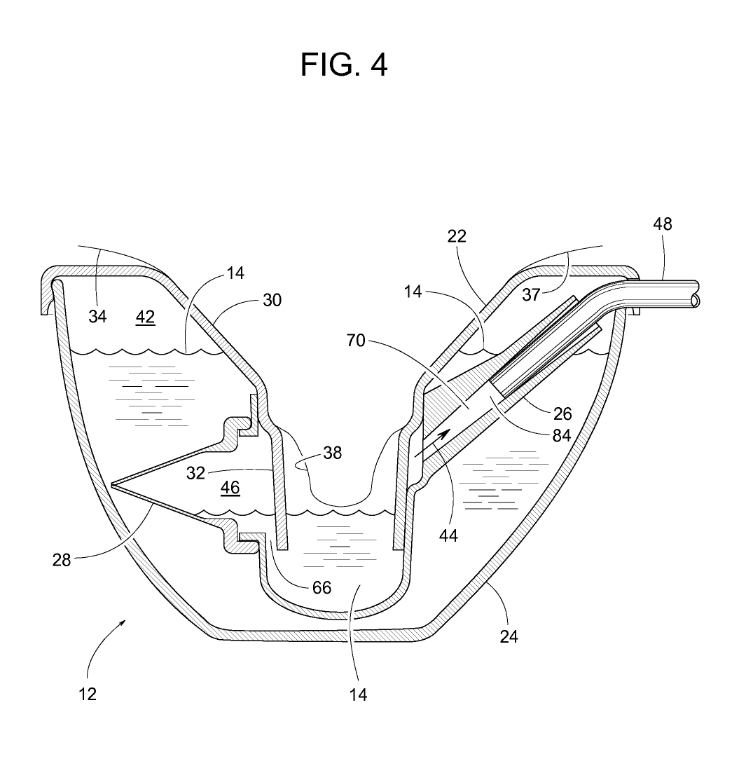

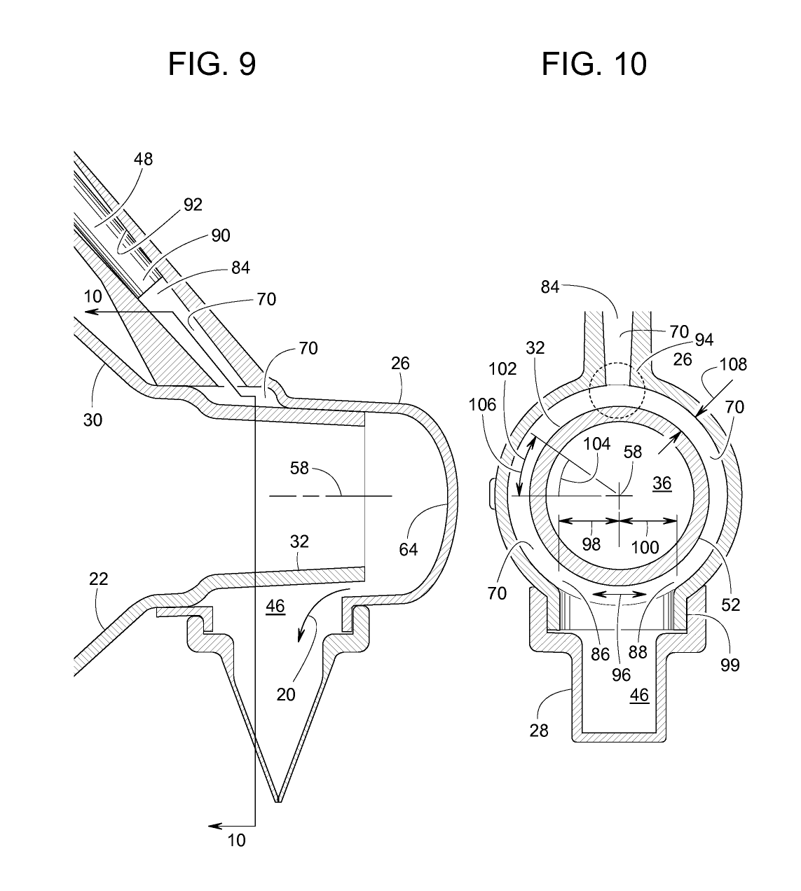

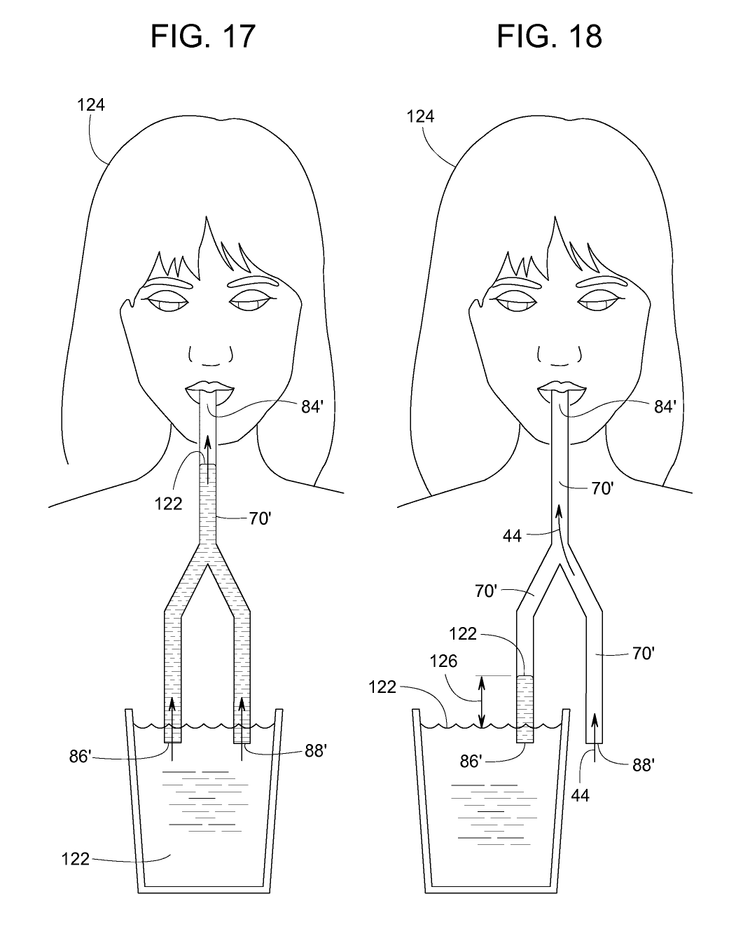

FIGS. 1-16 show various views of an example breast pump system 10 that includes a milk collection device 12 with means for preventing milk 14 from backflowing to a vacuum pump 16. FIGS. 17-19 illustrate the underlying operating principle of vacuum breakers. And FIGS. 21-22 show variations of the system design. The general design isolates a subatmospheric air flow path 102 (FIG. 10) from a milk flow path 20 (FIG. 9) even if milk collection device 12 it tipped completely over (FIG. 4). The vacuum breaker concept keeps fluids separated without using conventional baffles, which inherently have crevices that can be difficult to clean.

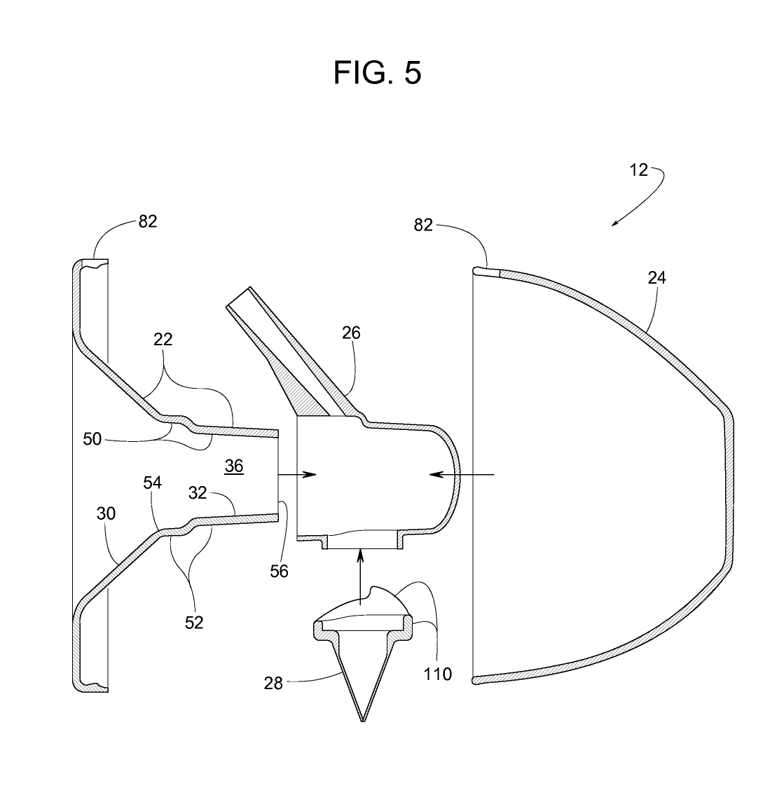

As an overview of the breast pump system's general construction, milk collection device 12 comprises four main parts: a funnel-shaped breast receiver 22, a domed outer shell 24, a fluid exchanger 26, and a unidirectional valve 28 (e.g., a check valve, a duckbill check valve, a reed valve, a ball check valve, a diaphragm check valve, a swing check valve, etc.). FIG. 1 shows these for main parts in an assembled operating state with the parts being positioned as a unit in a predetermined orientation, and FIG. 5 shows them in a disassembled cleaning state. Breast receiver 22 itself comprises a breast guide 30 and a nipple receptacle 32. Breast guide 30 is generally conical for fittingly receiving a breast 34 of a lactating woman 36, and nipple receptacle 32 is tubular and defines a nipple chamber 36 for receiving a nipple 38 of breast 34.

In some examples, outer shell 24 removably connects to a flange 40 of breast receiver 22 to define a milk storage chamber 42 between outer shell 24 and breast receiver 22. Fluid exchanger 26 is coupled to breast receiver 22 to provide means for strategically directing milk 14 and air 44 within milk collection device 12. Valve 28 establishes a milk charging chamber 46 between nipple receptacle 36 and storage chamber 42. In some examples, charging chamber 46 is cycled between positive and negative pressure to draw discrete quantities of expressed milk from nipple receptacle 36. During periods of positive pressure, charging chamber 46 discharges each discrete quantity or charge through valve 28 to storage chamber 42.

To provide charging chamber 46 with air 44 cyclically at subatmospheric pressure and positive or atmospheric pressure, a suction tube 48 couples milk collection device 12 to vacuum pump 16. The term, “vacuum pump,” refers to any device that provides subatmospheric pressure continuously, cyclically, or at least momentarily. Vacuum pump 16 is schematically illustrated to represent all types of vacuum pumps, examples of which include, but are not limited to, a diaphragm pump, a bellows pump, a piston pump, a reciprocating pump, a peristaltic pump, a positive displacement pump, a gear pump, a lobed rotor pump, a screw compressor, a scroll compressor, and a rotary vane pump.

The breast pump system's structure and operation can be further understood with additional definitions and explanations of some detailed features of the system. Nipple receptacle 36 has an inner curved wall surface 50, an outer curved wall surface 52, a proximate end 54 and a distal end 56. The nipple receptacle's tubular shape defines a longitudinal centerline 58 and nipple chamber 30. A minimum radial distance 60 exists between longitudinal centerline 58 and inner curved wall surface 50, wherein the minimum radial distance is measured perpendicular to centerline 58. Nipple receptacle 36 extends longitudinally in a forward direction 62 (parallel to centerline 58) from proximate end 54 to distal end 56. In some examples, nipple chamber 36 extends farther forward than distal end 56 of nipple receptacle 32; however, any part of nipple receptacle 32 that happens to extend farther forward than nipple chamber 36 is considered an extension beyond distal end 56 and thus is not considered the receptacle's distal end 56 itself. In some examples, the most forward point of nipple chamber 36 is at a domed concave surface 64 on fluid exchanger 26. Surface 64 being domed rather than flat makes fluid exchanger 26 easier to clean after fluid exchanger 26 is separated from breast receiver 22.

When breast receiver 22 and valve 28 are attached to fluid exchanger 26, the resulting assembly produces various fluid passages, chambers and sealing interfaces. Upon disassembly, the passages, chambers and sealing interfaces become more open for easier cleaning and sanitizing. Examples of such passages, chambers and sealing interfaces include charging chamber 46, nipple chamber 36, a milk passage 66 for conveying milk 14 from nipple chamber 36 to charging chamber 46, a valve outlet 68 that periodically discharges discrete volumes of milk 14 to storage chamber 42, an air duct 70 that connects suction tube 48 in fluid communication with charging chamber 46, a primary sealing interface 72, and a secondary sealing interface 74.

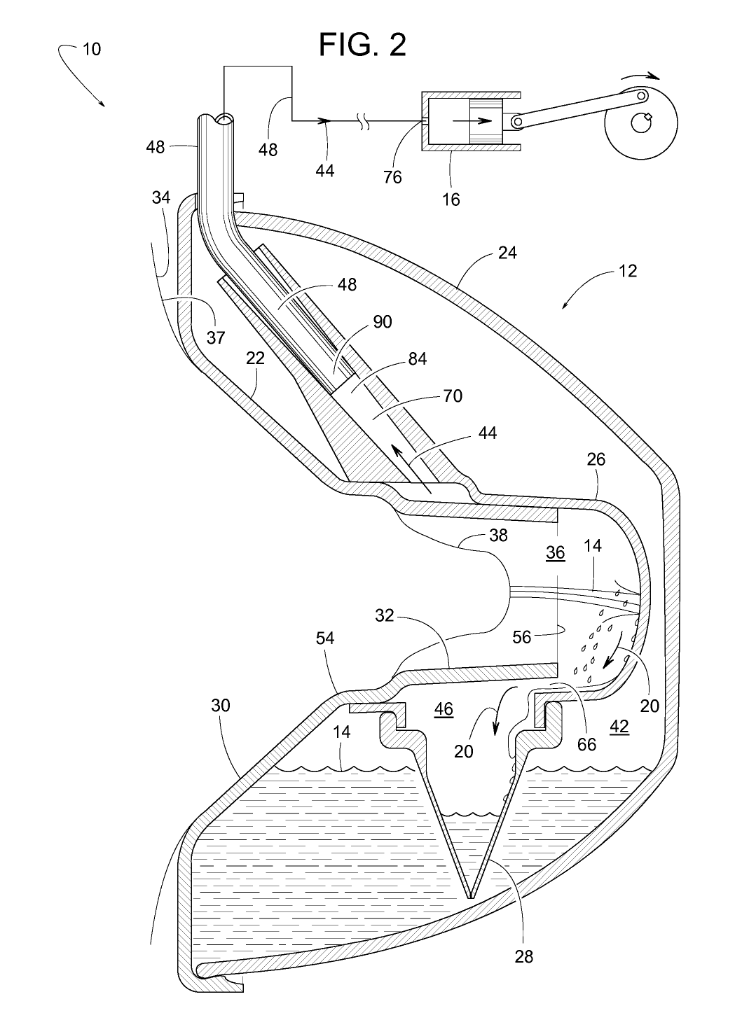

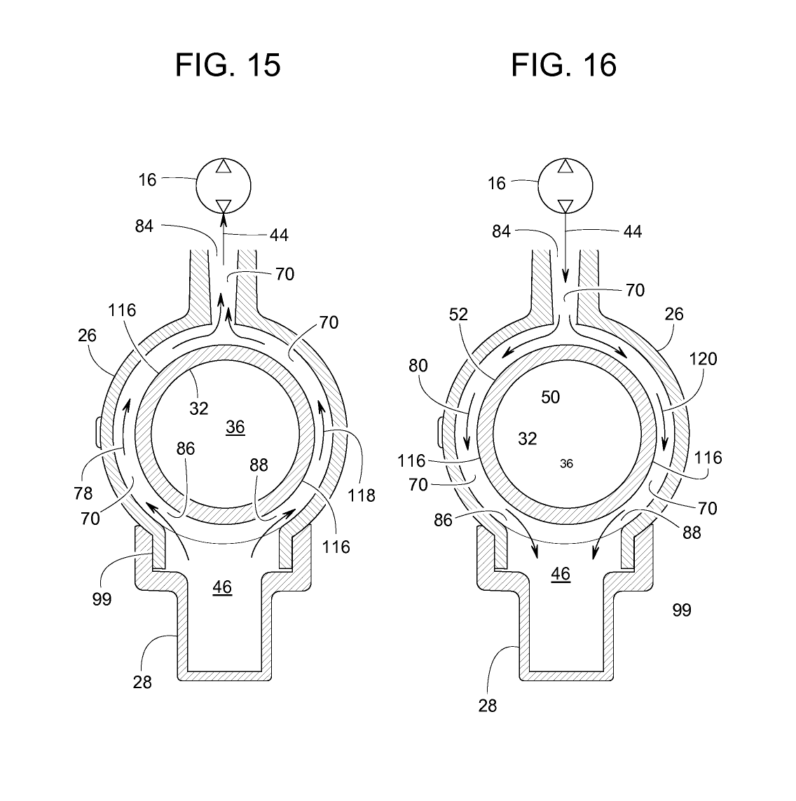

In some examples, system 10 operates in an alternating manner of suction periods and pressurized periods. During suction periods, as shown in FIGS. 2 and 15, vacuum pump 16 applies suction or air at subatmospheric pressure to a remote end 76 of suction tube 48. At least some of the vacuum reaches nipple chamber 36 to draw milk expressed from nipple 38. The expressed milk 14 flows from nipple chamber 36, flows through milk passage 66, and collects at the bottom of charging chamber 46. The negative air pressure produced by vacuum pump 16 creates a first current of air 78 (FIG. 15) that effectively moves from nipple chamber 36 and effectively flows in series through milk passage 66, through charging chamber 46, through air duct 70 (FIGS. 9, 10, 15 and 16), through suction tube 48, and to vacuum pump 16. The terms, “effectively moves” and “effectively flows” means that there is some air movement from an upstream point toward a downstream point, but the air at the upstream point will not necessarily reach the downstream point, due to the travel distance and/or other flow constraints.

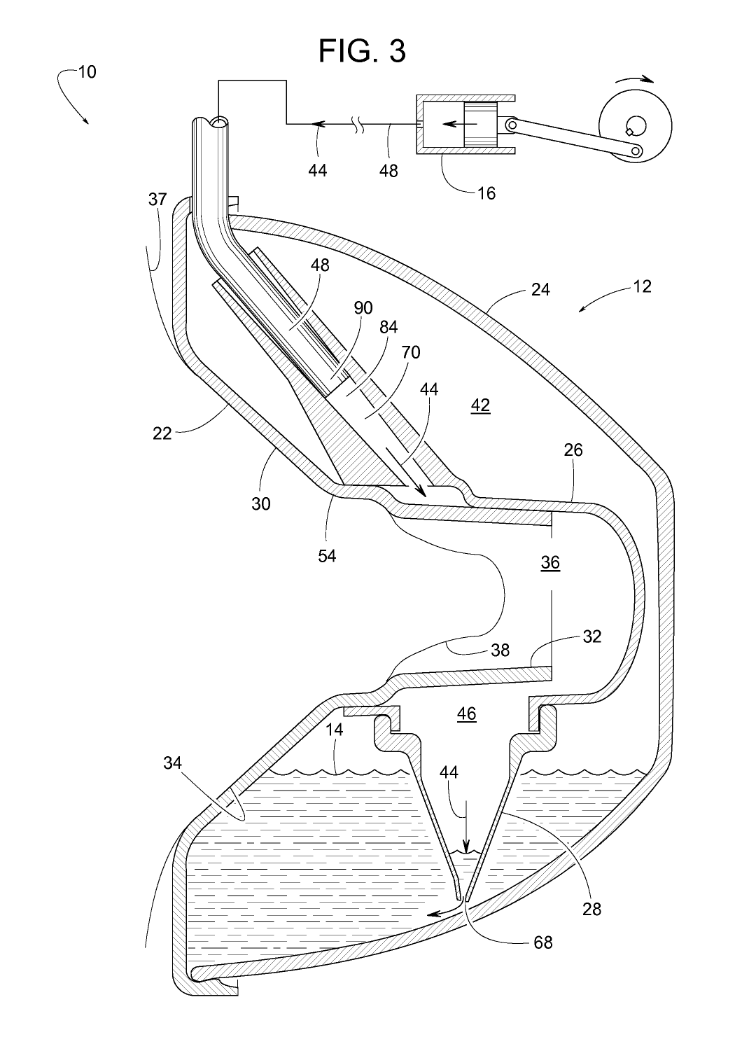

During pressurized periods, as shown in FIGS. 3 and 16, vacuum pump 16 applies positive air pressure to suction tube 48. The positive pressure creates a second current of air 80 that effectively flows in series through suction tube 48, through air duct 70, through milk passage 66, and into nipple chamber 36. The air pressure in charging chamber 46 forces milk 14 (collected during the previous suction period) from charging chamber 46, down through valve 28, and into storage chamber 42. The air pressure in nipple chamber 36 allows breast 34 to relax prior to the next suction period.

The alternating cycle of suction and pressure is repeated for as long as desired or until storage chamber 42 is filled to some predetermined capacity. Upon completion of the pumping process, any suitable means can be used for transferring collected milk from storage chamber 42 to a bottle or to some other convenient storage container. One example method for transferring milk 14 from storage chamber 42 is to pull suction tube 48 out from within an opening 82 (FIG. 5) between breast receiver 22 and outer shell 24, and then pour collected milk 14 out through opening 82. Another method is to turn milk collection device 12 over (e.g., FIG. 4), remove breast receiver 22 from outer shell 24, and simply pour milk 14 out from shell 24.

Although FIG. 4 is referred to illustrate means for emptying milk 14 collected in storage chamber 42, the primary purpose of FIG. 4 is to show how well device 12 tolerates a completely tipped-over condition while still preventing milk 14 from backflowing into suction tube 48. Device 12 has three features that prevent milk backflow. One, in the tipped-over position, air duct 70 remains elevated above milk passage 66. Two, a circumferential seal 74 (FIG. 12) exists between air duct 70 and milk 14 in nipple chamber 36. Three, air duct 70 connects to charging chamber 46 at two spaced apart openings 86 and 88 (see FIG. 15 and the explanation referencing FIGS. 17, 18 and 19)

Preventing milk 14 from entering suction tube 48 is important for several reasons. Milk droplets or even a milk film trapped inside a narrow suction tube can be very difficult to thoroughly clean and sanitize. If left unclean, the trapped milk might contaminate future milk collections. Also, if milk in suction tube 48 migrates into vacuum pump 16, the milk can be even more difficult to remove and can possibly damage or destroy pump 16. Tolerating such unsanitized conditions is generally unheard of in the fields of medicine and food processing.

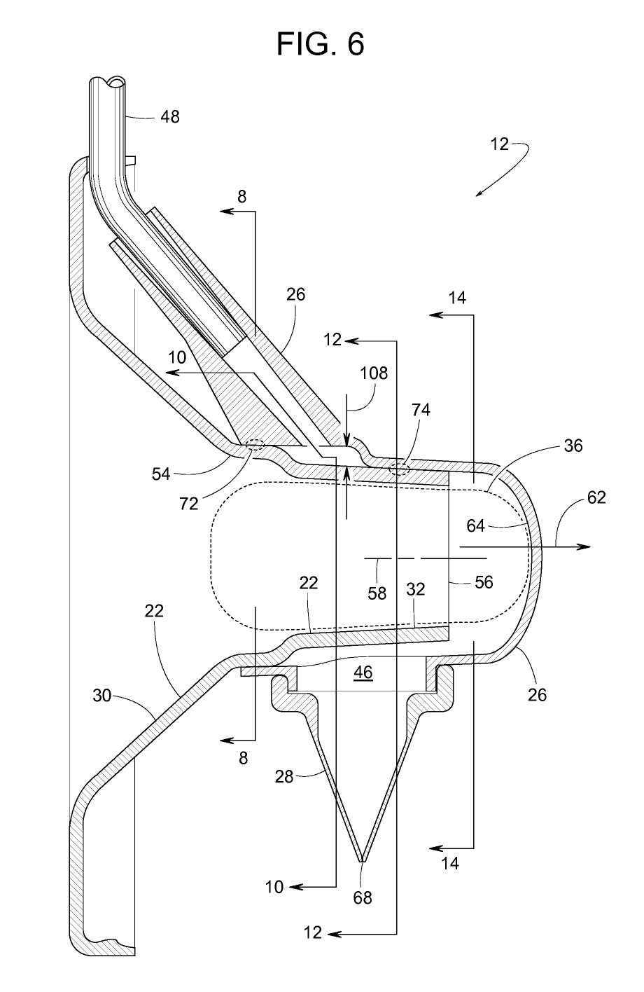

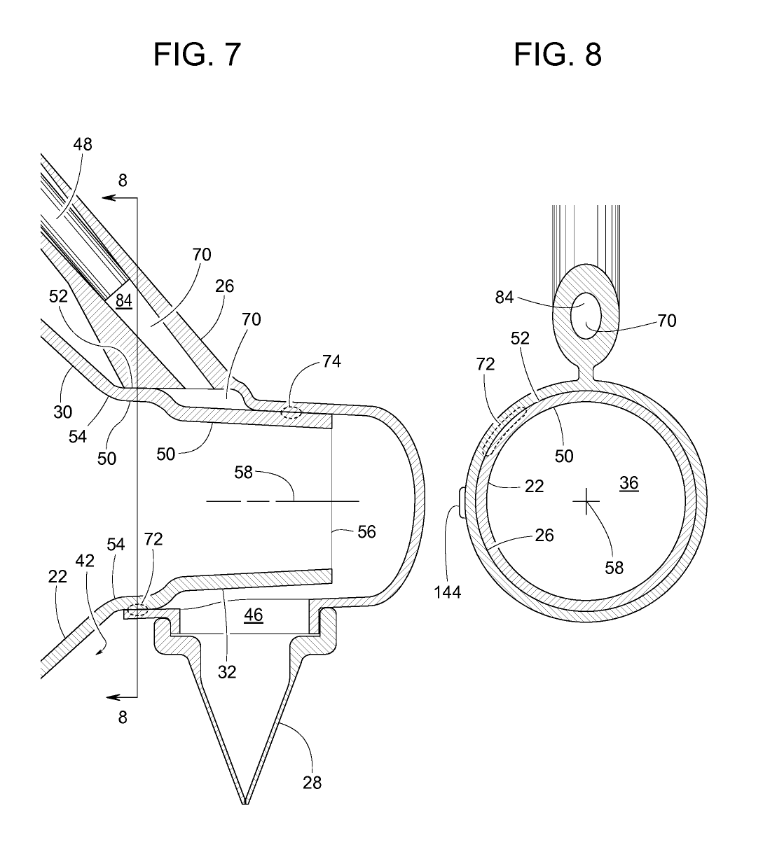

FIG. 6 serves as somewhat of an index drawing for a subsequent series of cross-sectional views. The views in the series are shown in sets of two and are identified as FIGS. 7-8, FIGS. 9-10, FIGS. 11-12, and FIGS. 13-14. FIGS. 7-8 show primary sealing interface 72 between an outer diameter of breast receiver 22 and an inner diameter of fluid exchanger 26. Primary sealing interface 72 is a relatively tight seal that extends 360 degrees circumferentially around centerline 58 to isolate localized pressure or vacuum within charging chamber 46 while the surrounding storage chamber 42 is at atmospheric pressure. In some examples, to ensure a positive seal, interface 72 tapers at 3-degrees in a lengthwise direction with reference to centerline 58.

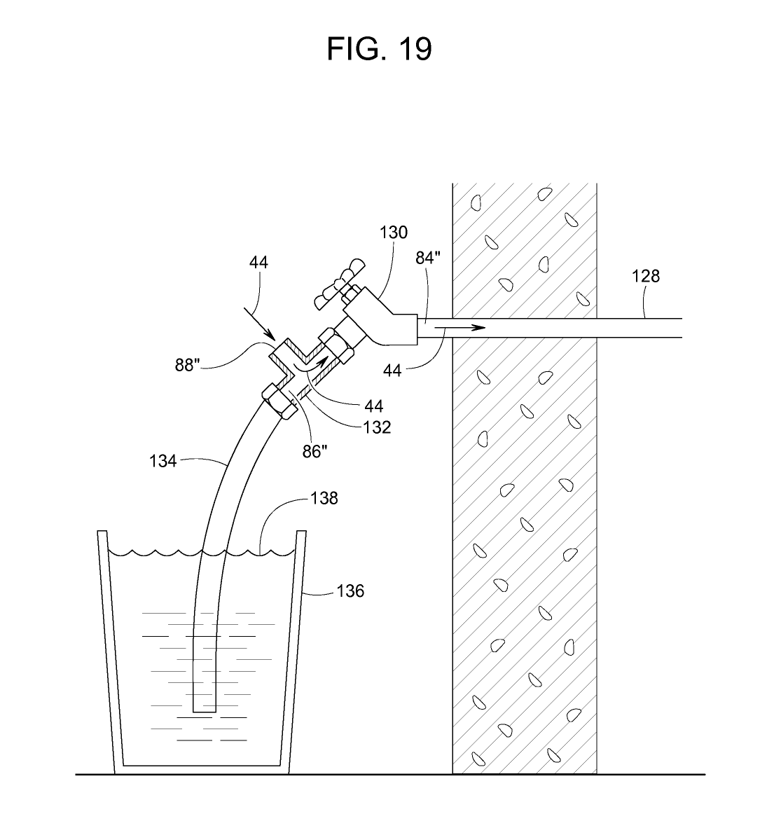

FIGS. 9-10 show one example of air duct 70 connecting vacuum tube 48 in fluid communication with charging chamber 46. In this example, air duct 70 comprises a supply port 84 at a connection end 90 of suction tube 48, a first opening 86 at charging chamber 46, and a second opening 88 at charging chamber 46. To connect tube 48 to supply port 84, connection end 90 of suction tube 48 press-fits into a tapered bore 92 of fluid exchanger 26. A fork 94 (e.g., one path leading to two) in air duct 70 connects supply port 84 in fluid communication with openings 86 and 88. Features 84, 86 and 88 of FIG. 10 correspond respectively to points 84′, 86′ and 88′ of FIG. 18. Features 84, 86 and 88 of FIG. 10 also correspond respectively to points 84″, 86″ and 88″ of FIG. 19.

To apply the “vacuum break” concept illustrated in FIGS. 17 and 18, fork 94 straddles nipple receptacle 36 so that openings 86 and 88 are spaced apart in a lateral direction 96 with the nipple receptacle longitudinal centerline 58 being laterally interposed between openings 86 and 88 (dimensions 98 and 100). In some examples, nipple receptacle 36 is flanked by openings 86 and 88, which means that the nipple's longitudinal centerline 58 is laterally between openings 86 and 88, as shown in FIG. 10. The spaced-apart distance and elevation of openings 86 and 88 can be increased by increasing the diameter of a flange 99 to which valve 28 is attached.

Still referring to FIG. 10, some examples of air duct 70 define a flow path 102 from supply port 84 to first opening 86, wherein a curved section of flow path 102 extends circumferentially an angular distance 104 of at least thirty degrees to avoid having to create an alternate flow path in front of or through nipple chamber 36. In some examples, at least one section 106 of flow path 102 lies within a radial gap 108 between fluid exchanger 26 and the nipple receptacle's outer curved wall surface 52. Upon disassembling device 12 to its disassembled cleaning state (FIG. 5), section 106 of flow path 102 is split apart, which makes flow path 102 and air duct 70 much more accessible for cleaning.

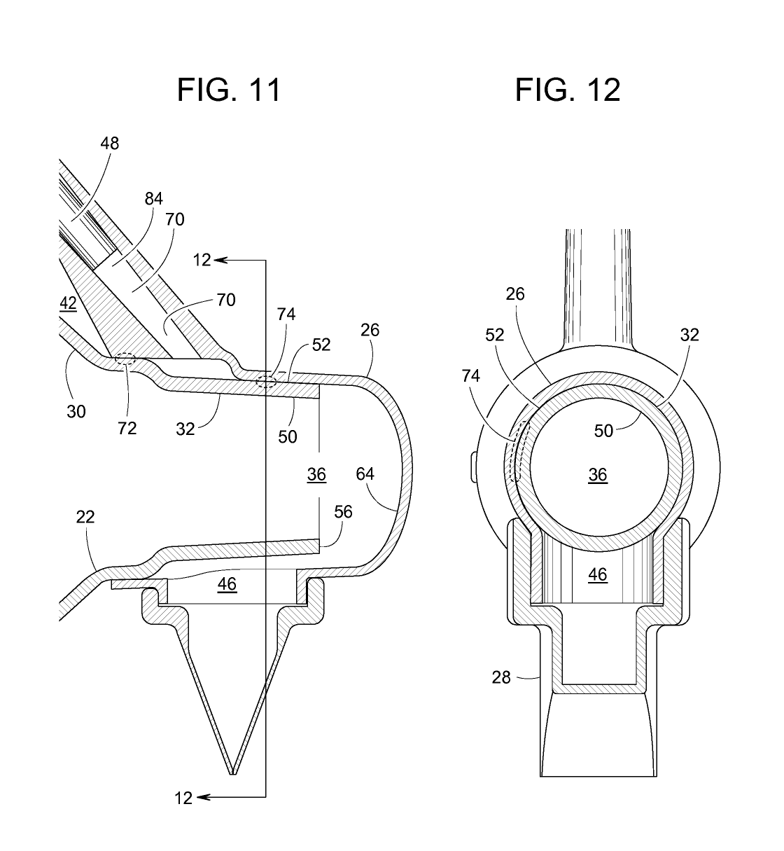

FIGS. 11 and 12 show secondary sealing interface 74 radially between fluid exchanger 26 and the nipple receptacle's outer curved wall surface 52. Secondary sealing interface 74 provides a barrier that prevents milk 14 from flowing directly from nipple chamber 36 to air duct 70. FIG. 11 shows air duct 70 being between primary sealing interface 72 and secondary sealing interface 74.

Primary sealing interface 72 is the more critical seal of the two because primary sealing interface 72 is subjected to an appreciable pressure differential between supply port 84 and storage chamber 42. Secondary sealing interface 74, however, is not as critical because the pressure differential between supply port 84 and nipple chamber 36 is nearly zero. Consequently, in some examples, primary sealing interface 72 is made to be a tighter seal than secondary sealing interface 74. In other words, when breast receiver 22 is snugly inserted into fluid exchanger 26, the radial forces at primary sealing interface 72 is greater than that at secondary sealing interface 74.

It can be important to have primary sealing interface 72 be the dominant seal because when breast receiver 22 is inserted into fluid exchanger 26, something has to “bottom out” first to stop the relative insertion movement of breast receiver 22 into fluid exchanger 26. If secondary sealing surface 74 or distal end 56 abutting domed surface 64 were to be the first parts to bottom out, that might leave some radial clearance or leak path at primary sealing interface 72. Intentionally making primary sealing interface 72 be the first to bottom out, loosens the manufacturing tolerances at other near bottom-out locations, thus increasing assembly reliability, reducing tooling costs, and simplifying manufacturing.

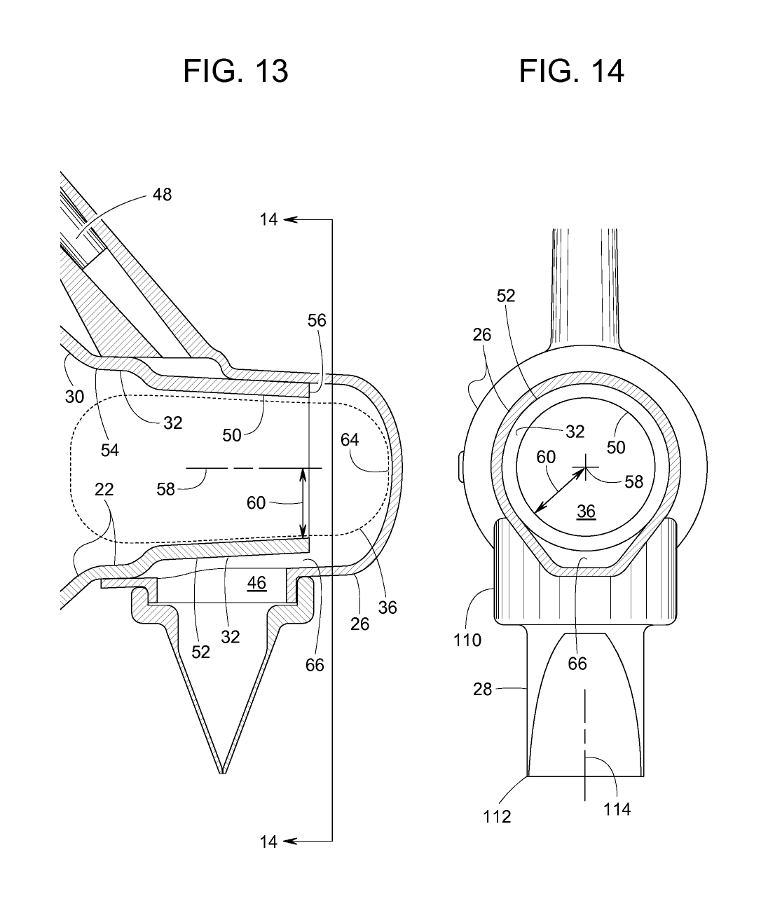

FIGS. 13 and 14 show milk passage 66 between charging chamber 46 and nipple chamber 36. FIGS. 14 and 5 show how an irregular shaped upper flange 110 of valve 28 serves as a means for “clocking” or rotationally aligning valve 28 to fluid exchanger 26. Such alignment can be important to avoid interference between a lower end 112 of valve 28 and outer shell 24. For instance, if valve 28 were rotated ninety degrees (about a vertical axis 114) from the position shown in FIG. 1, the valve's lower end 112 might press up against outer shell 24, whereby outer shell 24 might hold valve 28 open and prevent it from closing.

FIGS. 15 and 16 illustrate an example breast pump method operating during a first suction period (FIGS. 2 and 15) and a second pressure period (FIGS. 3 and 16). FIG. 15 shows during the first period, directing first current of air 78 in a first curved upward direction circumferentially across a first outer convex wall surface 116 of nipple receptacle 32. FIG. 15 also shows during the first period, directing a third current of air 118 in a second curved upward direction circumferentially across the nipple receptacle's first outer convex wall surface 116. FIG. 16 shows during the second period, directing second current of air 80 in a first curved downward direction circumferentially across the nipple receptacle's first outer curved wall surface 116. FIG. 16 also shows during the second period, directing a fourth current of air 120 in a second curved downward direction circumferentially across the nipple receptacle's first outer curved wall surface 116, wherein nipple receptacle 32 is interposed between first current of air 78 and third current of air 118 during the first period, and nipple receptacle 32 is interposed between second current of air 80 and fourth current of air 120 during the second period.

FIGS. 17 and 18 illustrates the concept of a vacuum breaker as a means for preventing a liquid 122 from backflowing up to a suction source 124. Liquid 122 only reaches suction source 124 when both openings 86′ and 88′ are submerged in liquid 122, as shown in FIG. 17. If only one opening 86′ is submerged and the other opening 88′ is exposed to air 44, as shown in FIG. 18, air 44 readily supplies the volume drawn in by suction source 124. Through a given opening, air can flow about thirty times easier than water. Consequently, only a slight pressure differential is needed for air 44 to rush through opening 88′ to suction source 124. That slight pressure differential creates only a slight pressure head 126 that is unable to lift liquid 122 from opening 86′ to suction source 124.

FIG. 19 provides another example of illustrating a vacuum breaker concept. This example involves the use of a residential water line 128, an outdoor faucet 130, a simplified vacuum breaker 132, and a garden hose 134 partially submerged in a bucket 136 of contaminated water 138. In this example, if unusual adverse conditions create a vacuum in water line 128, clean outdoor air 44 rather than contaminated water 138 will be drawn into water line 128.

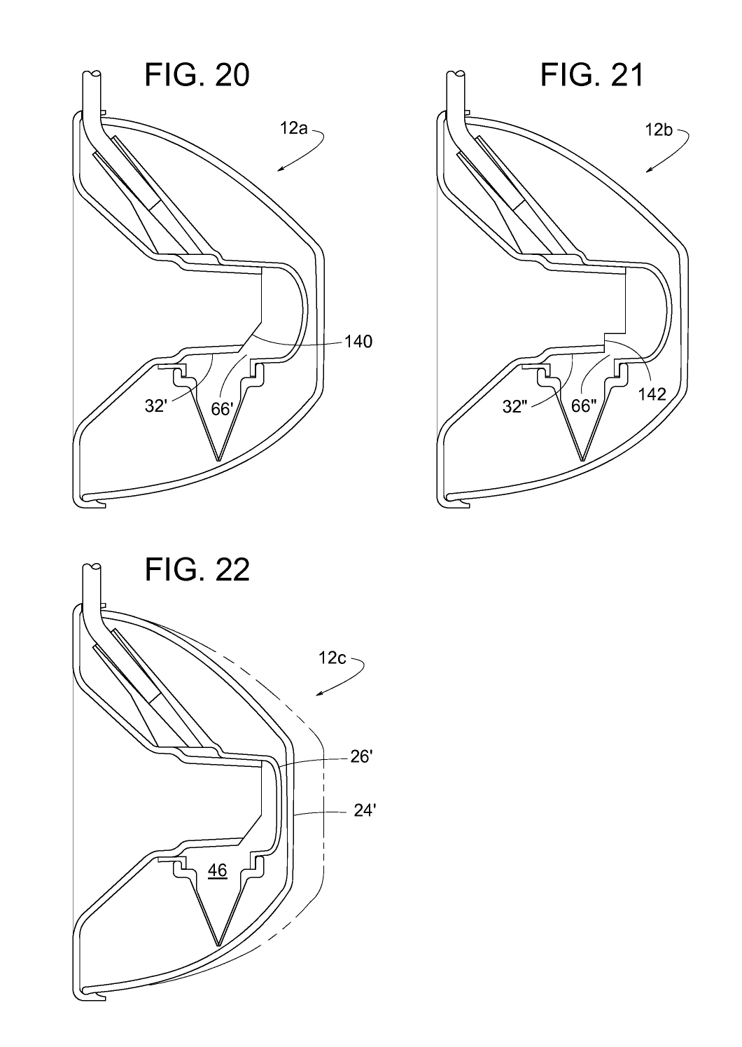

FIGS. 20, 21 and 22 show various design modifications. FIG. 20 shows an altered milk passage 66′ created by a beveled edge 140 at the end of a nipple receptacle 32′. FIG. 21 shows an altered milk passage 66″ created by a notched edge 142 at the end of a nipple receptacle 32″. FIG. 22 shows that a stubbier fluid exchanger 26′ and a less protruding outer shell 24′ can be used when air duct 4 curves around the sides of the nipple receptacle rather than in front of it. The stubbier fluid exchanger 26′ also reduces the effective volume of charging chamber 46, which can be beneficial when using certain low displacement vacuum pumps.

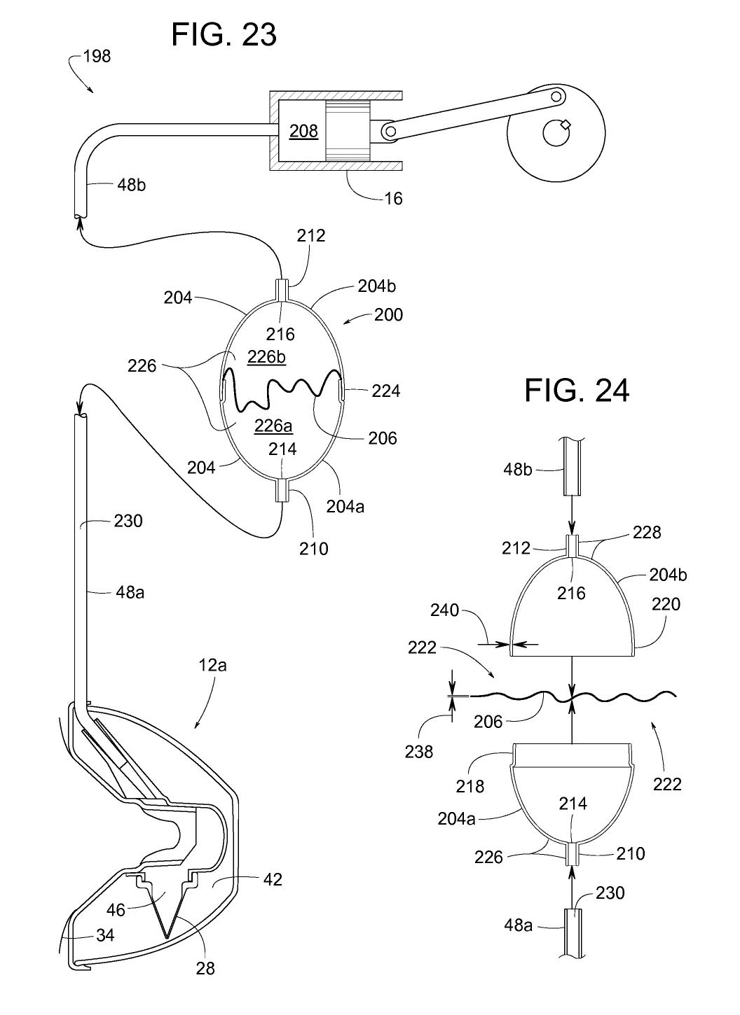

FIGS. 23-29 show an example breast pump system 198 with an example fluid isolator 200 for preventing milk 14 in a milk collection device (e.g., milk collection device 12, 12a, 12b, 202, etc.) from backflowing to a vacuum pump (e.g., vacuum pump 16 or 16′). Fluid isolator 200 comprises a shell 204 with an internal diaphragm 206 that prevents fluid in the milk collection device's charging chamber 46 or 46′ from backflowing into an air chamber 208 (or air chamber 208′) of vacuum pump 16 (or pump 16′). Diaphragm 206, however is sufficiently pliable and limp to convey pressure and volume changes in air chamber 208 to comparable pressure and volume changes in charging chamber 46.

In some examples, fluid isolator 200 comprises a first shell 204a, a second shell 204b, diaphragm 206, a first tubular fitting 210, and a second tubular fitting 212. First shell 204a has a first port 214 extending through first tubular fitting 210, and second shell 204b has a second port 216 extending through second tubular fitting 212. In some examples, as shown in FIG. 24, fluid isolator 200 is assembled by clamping or sandwiching diaphragm 206 between mating rim edges 218 and 220 of shells 204a and 204b respectively, whereby a peripheral portion 222 of diaphragm 206 becomes sealingly pinched at a joint 224 between shells 204a and 204b. In some examples joint 224 is an interlocking connection between shells 204a and 204b. In some examples joint 224 is an interference press fit between shells 204a and 204b. In some examples, joint 224 is further sealed with adhesive or ultrasonic welding. After assembly, surplus material of the diaphragm's peripheral portion 222 can be trimmed off.

Once assembled, in some examples, a first suction tube 48a is attached to first tubular fitting 210, a second suction tube 48b is attached to second tubular fitting 212, and opposite ends of suction tubes 48a and 48b are attached respectively to milk collection device 12a and vacuum pump 16. The assembly of first and second shells 204a and 204b creates an assembled shell 204 that has an internal volume 226. Diaphragm 206 divides internal volume 226 into a first chamber 226a within first shell 204a and a second chamber 226b within second shell 204b. First port 214 connects first chamber 226a in fluid communication with charging chamber 46, and second port 216 connects second chamber 226b in fluid communication with the vacuum pump's air chamber 208.

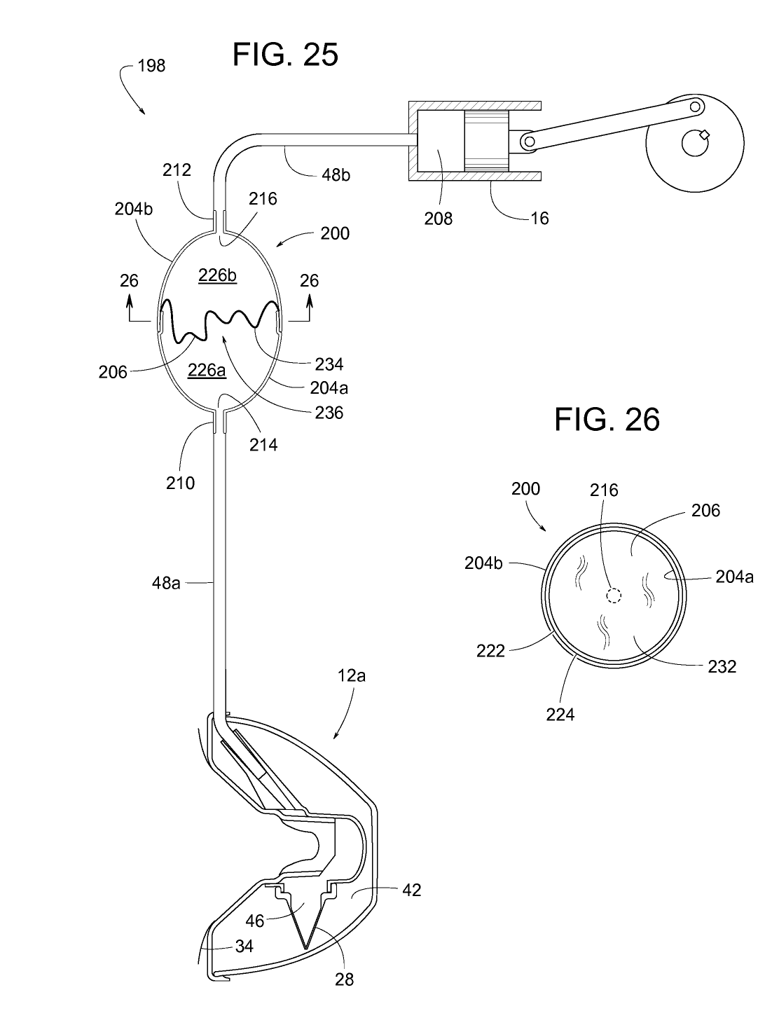

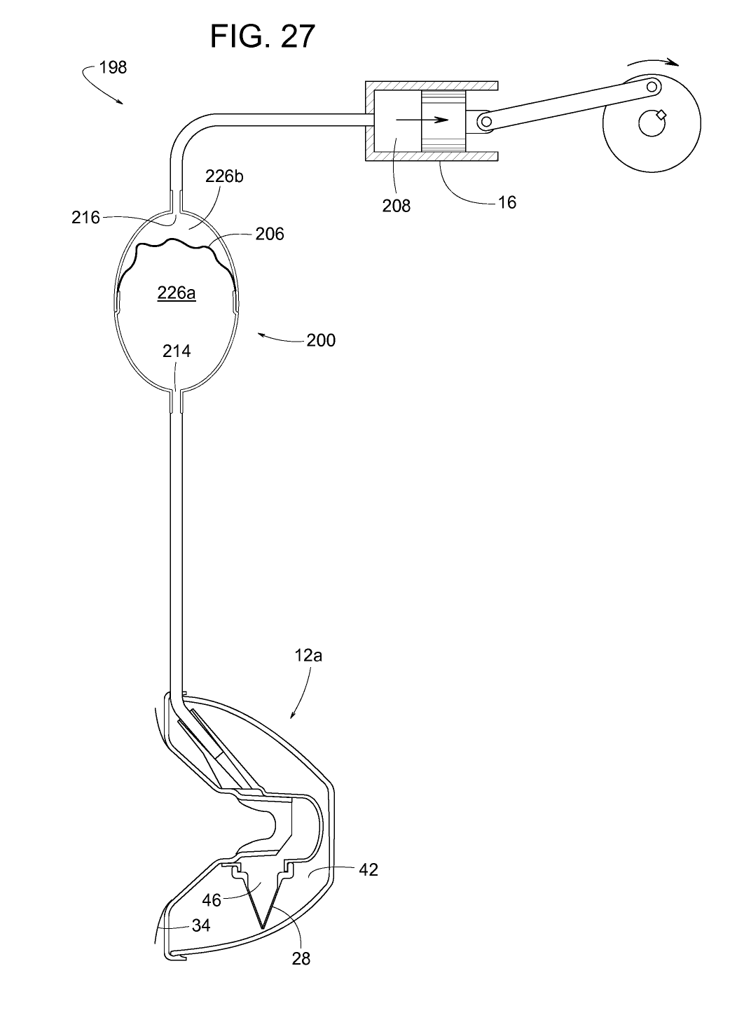

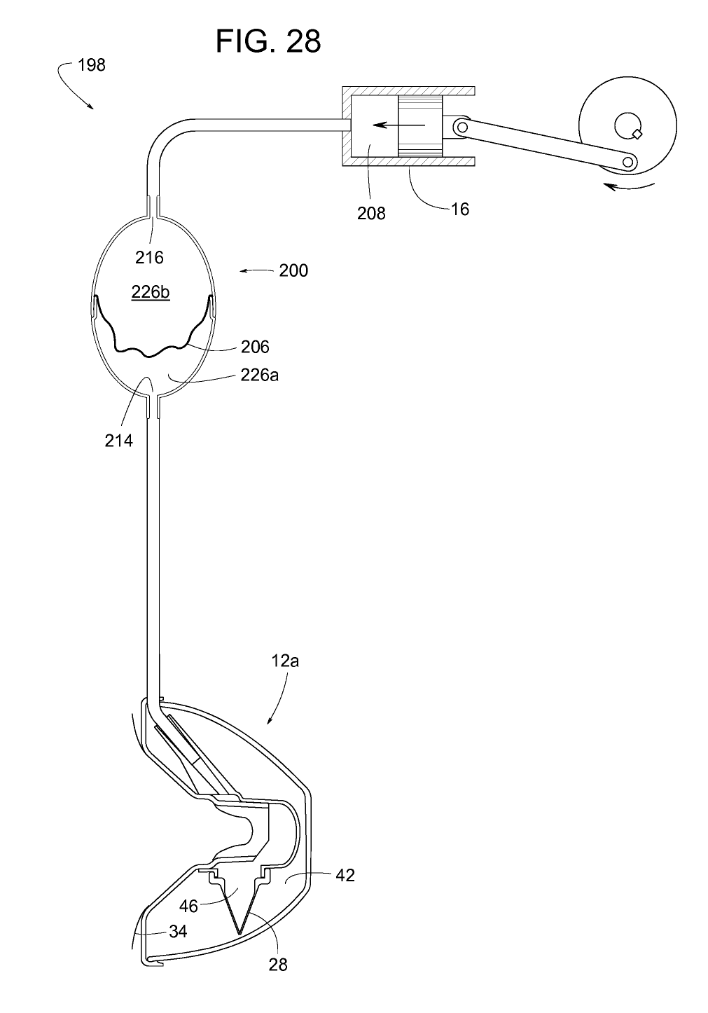

As explained earlier with reference to FIGS. 1-22, vacuum pump 16 operates cyclically between a negative pressure state (FIGS. 2 and 27) and a positive pressure state (FIGS. 3 and 28). FIG. 25 shows vacuum pump 16 in a neutral or non-operating state, wherein chambers 226a and 226b are at atmospheric pressure. Air in the vacuum pump's air chamber 208 and in the isolator's second chamber 226b is at a first negative pressure when vacuum pump 16 is in the negative pressure state. The negative pressure in the isolator's second chamber 226b shifts diaphragm 206 toward second port 216, as shown in FIG. 27. Conversely, air in the vacuum pump's air chamber 208 and in the isolator's second chamber 226b is at a first positive pressure when vacuum pump 16 is in the positive pressure state. Positive pressure in the isolator's second chamber 226b shifts diaphragm 206 toward first port 214, as shown in FIG. 28.

For most, if not all, of the vacuum pump's periods of positive and negative pressure (e.g., all except for perhaps the very ends of each pump cycle), the pliability of diaphragm 206 allows diaphragm 206 to remain substantially limp and unstressed, as shown in FIGS. 27 and 28. Thus, the pressure on both sides of diaphragm 206 is substantially equal, so the pressure in first chamber 226a generally equals the pressure in second chamber 226b as pump 16 cycles. Consequently, fluid isolator 200 effectively transmits the varying air pressure in the vacuum pump's air chamber 208 to the milk collection device's charging chamber 46.

Some examples of fluid isolator 200 include one or more features that enhance the performance and usefulness of fluid isolator 200. For instance, in some examples, shell 204a and/or 204b is made of a see-through material (e.g., clear, tinted, transparent, translucent ABS or other plastic material). This allows a user to readily observe the action of diaphragm 206 as a means for evaluating how well fluid isolator 200 and the rest of system 198 is operating. In some examples, shells 204a and 204b are domed (e.g., spherical, parabolic, etc.) to accommodate the expanded shape of diaphragm 206 during the end of each pump cycle and to reduce the overall size of shell 204. In some examples, shell 204 has a spherical or oblong shape, mechanical interlocking joint, and material composition similar to that of a conventional two-piece hollow plastic egg, commonly known as a, “plastic Easter egg.” In some examples, first tubular fitting 210 encircling first port 214 is a seamless integral extension of first shell 204a (e.g., they are produced in the same plastic injection mold) such that first tubular fitting 48 and first shell 204a provide a first seamless unitary piece 226. Likewise, in some examples, second tubular fitting 212 encircling second port 216 is a seamless integral extension of second shell 204b (e.g., they are produced in the same plastic injection mold) such that second tubular fitting 212 and second shell 204b provide a second seamless unitary piece 228.

To ensure that fluid isolator 200 has the volumetric capacity to handle the demand that milk collection device 12a places upon it, the cumulative internal volume 226 between shells 204a and 204b is greater than the milk collection device's charging chamber 46. To accommodate an inner tube volume 230 of first suction tube 48a, some examples of fluid isolator 200 have the cumulative internal volume 226 between shells 204a and 204b be greater than inner tube volume 230, wherein inner tube volume 230 equals the internal open cross-sectional area of suction tube 48a times the tube's full length from first port 214 to milk collection device 12a.

In some examples, diaphragm 206 is of a size that ensures that diaphragm 206 stays limp throughout at least most of the pump's cycle. More specifically, in some examples, joint 224 encircles a cross-sectional area 232 of internal volume 226, and the side of diaphragm 206 that faces first shell 204a has a surface area 234 that is greater than cross-sectional area 232. Consequently, a central portion 236 of diaphragm 206 is pliable and limp within the fluid isolator's internal volume 226 when both the first chamber 226a and second chamber 226b contain air at atmospheric air pressure, as shown in FIGS. 23 and 25. The diaphragm's central portion 236 is also shown in a relaxed position that is neither biased toward first shell 204a nor biased toward second shell 204b. In some examples when diaphragm 206 is in the relaxed position, central portion 236 is generally halfway between ports 214 and 216. To provide diaphragm 206 with sufficient pliability while shell 204 is relatively rigid, some examples of fluid isolator 200 have a diaphragm material thickness 238 that is less than a shell material thickness 240 of shell 204a and/or 204b. In some examples, diaphragm 206 is made of MYLAR, which is a registered trademark of Dupont Teijin Films of Wilmington, Del.

In some examples, fluid isolator 200 is used as an aftermarket product that can be added to almost any known breast pump system including, but not limited to, FREEMIE style breast pump systems and MEDELA style breast pump systems, wherein FREEMIE is a registered trademark of DAO Health of Sacramento, Calif., and MEDELA is a registered trademark of Medela Holding AG of Barr, Switzerland. In some examples, fluid isolator 200 is added by cutting the breast pump system's existing suction tube 48 and connecting the cut ends of the tube to ports 214 and 216.

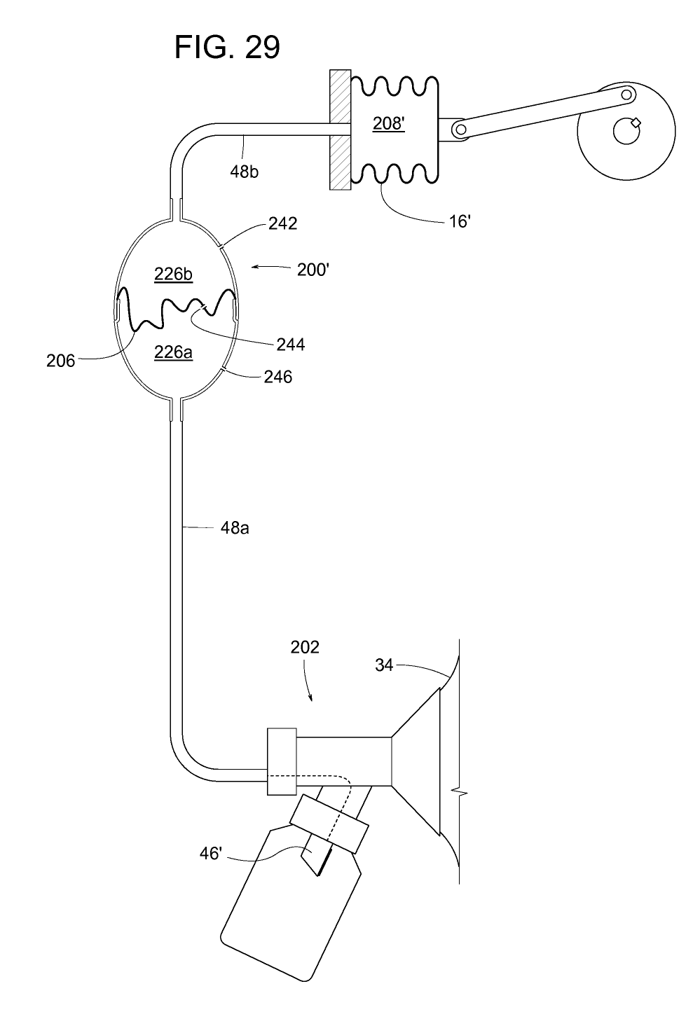

FIG. 29, for instance, shows fluid isolator 200′ installed between a MEDELA style vacuum pump 16′ (e.g., a bellows pump) and a MEDELA style milk collection device 202. In this example, first suction tube 48a connects fluid isolator 200′ to milk collection device 200, and a second similar suction tube 48b connects fluid isolator 200′ to vacuum pump 16′.

In addition or alternatively, some examples of fluid isolator 200′ (or fluid isolator 200) have a tiny supplementary opening 242 that connects second chamber 226b in fluid communication with atmospheric air. A small air leakage through supplementary opening 242 provides means for synchronizing or properly coordinating the position of diaphragm 206 with the cyclical periods of vacuum pump 16′. Supplementary opening 242 is sufficiently small to create an inconsequential loss in the breast pump system's operating efficiency. In some examples, supplementary opening 242 provides a fluid flow resistance equivalent to or less than that of a 0.5 mm diameter orifice.

In addition or alternatively, some examples of fluid isolator 200′ have a tiny supplementary opening 244 that connects first chamber 226a in fluid communication with second chamber 226b. A small air leakage through supplementary opening 244 provides means for synchronizing or properly coordinating the position of diaphragm 206 with the cyclical periods of vacuum pump 16′. Supplementary opening 244 is sufficiently small to create an inconsequential loss in the breast pump system's operating efficiency. In some examples, supplementary opening 244 provides a fluid flow resistance equivalent to or less than that of a 0.5 mm diameter orifice.

In addition or alternatively, some examples of fluid isolator 200′ have a tiny supplementary opening 246 that connects first chamber 226a in fluid communication with atmospheric air. A small air leakage through supplementary opening 226a provides means for injecting a small volume of air between first chamber 226a and charging chamber 46′. If a milk droplet were to accidentally backflow into first suction tube 48a, the injected small volume of air serves to push the droplet back toward charging chamber 46′. Supplementary opening 246 is sufficiently small to create an inconsequential loss in the breast pump system's operating efficiency. In some examples, supplementary opening 246 provides a fluid flow resistance equivalent to or less than that of a 0.5 mm diameter orifice.

For further clarification, the term, “suction tube” refers to any conduit having a tubular wall of sufficient thickness, stiffness, and/or strength to convey air at subatmospheric pressure. In some examples, suction tube 48 is more flexible than outer shell 24, breast receiver 22, and/or fluid exchanger 26. Such tube flexibility makes tube 48 easier to use and fit to fluid exchanger 26. The term, “coupled to” refers to two members being connected either directly without an intermediate connecting piece or being connected indirectly via an intermediate connecting piece between the two members. The term, “coupled to” encompasses permanent connections (e.g., bonded, welded, etc.), seamless connections (e.g., the two members are of a unitary piece), and separable connections. The term, “opening” of a fluid pathway refers to a cross-sectional area through which fluid is directed to flow in a direction generally perpendicular to the area as guided by the fluid pathway. The term, “radial gap” refers to clearance as measured in a direction perpendicular to longitudinal centerline 58. The terms, “negative pressure,” “subatmospheric pressure,” and “vacuum” all refer to a pressure that is less than atmospheric pressure. The term, “positive pressure,” refers to a pressure that is greater than atmospheric pressure. The term, “gage pressure” refers to pressure relative to atmospheric air pressure. Storage chamber 42 is not necessarily for long term storage but rather for collecting and temporarily storing milk 14 as the lactating woman is expressing milk. In some examples, milk collection device 12 includes a slot-and-key 144 alignment feature (FIG. 8) that establishes a certain desired rotational alignment (about longitudinal centerline 58) between fluid exchanger 26 and breast receiver 22. In some examples, the positive pressure within air chamber 204 is only sufficiently positive to force air lightly through suction tube 48 leading from vacuum pump system 202 to milk collection device 12a. The term, “pliable” at it refers to a diaphragm means that the diaphragm is sufficiently flexible to be crumpled and subsequently restored to its original shape prior to being crumpled. The term, “limp” as it refers to a diaphragm means that a central portion of the diaphragm is unstressed (i.e., neither in tension nor in compression). Although fluid isolators 200 and 200′ has been described with reference to some example vacuum pumps and milk collection devices, fluid isolators 200 and 200′ can be readily used with many other types of vacuum pumps and milk collection devices.

Although the invention is described with respect to a preferred embodiment, modifications thereto will be apparent to those of ordinary skill in the art. The scope of the invention, therefore, is to be determined by reference to the following claims:

1. A breast pump system that uses air for assisting a lactating woman in collecting milk expressed by a breast of the lactating woman, the breast pump system comprising:
a vacuum pump defining an air chamber, the vacuum pump being operable cyclically between a negative pressure state and a positive pressure state, the air in the air chamber being at a first negative pressure when the vacuum pump is in the negative pressure state, the air in the air chamber being at a first positive pressure when the vacuum pump is in the positive pressure state;
a milk collection device being configured to fittingly receive the breast of the lactating woman, the milk collection device defining a charging chamber that is in fluid communication with the breast when the breast fittingly engages the milk collection device;
a first suction tube being connected to the milk collection device and being connected in fluid communication with the charging chamber; and
a fluid isolator coupling the first suction tube to the vacuum pump, the fluid isolator comprising a first shell, a second shell, and a diaphragm; the first shell defining a first port and a first chamber; the second shell defining a second port and a second chamber; the first shell being coupled to the second shell at a joint to define an internal volume between first shell and the second shell; the diaphragm extending across the internal volume and being connected to at least one of the first shell and the second shell at the joint; the diaphragm providing a seal between the first chamber and the second chamber; the first port connecting the first chamber in fluid communication with the first suction tube; and the second port connecting the second chamber in fluid communication with the air chamber of the vacuum pump.

2. The breast pump system of claim 1, wherein at least one of the first shell and the second shell comprises a see-through material.

3. The breast pump system of claim 1, wherein both the first shell and the second shell are domed.

4. The breast pump system of claim 1, further comprising a second suction tube that couples the second shell to the vacuum pump.

5. The breast pump system of claim 1, wherein a central portion of the diaphragm is pliable and limp within the internal volume of the fluid isolator when both the first chamber and the second chamber contain air at atmospheric air pressure.

6. The breast pump system of claim 1, wherein a central portion of the diaphragm has a relaxed position that is neither biased toward the first shell nor biased toward the second shell.

7. The breast pump system of claim 1, wherein the diaphragm has a diaphragm material thickness, at least one of the first shell and the second shell has a shell material thickness, and the diaphragm material thickness is less than the shell material thickness.

8. The breast pump system of claim 1, wherein a peripheral portion of the diaphragm is pinched at the joint between the first shell and the second shell.

9. The breast pump system of claim 1, wherein the joint circumscribes a cross-sectional area of the internal volume, the diaphragm has a surface area facing the first shell, and the surface area is greater than the cross-sectional area of the internal volume.

10. The breast pump system of claim 1, further comprising:
a first tubular fitting encircling the first port, the first tubular fitting extending integrally from the first shell such that the first tubular fitting and the first shell provide a first seamless unitary piece; and
a second tubular fitting encircling the second port, the second tubular fitting extending integrally from the second shell such that the second tubular fitting and the second shell provide a second seamless unitary piece.

11. The breast pump system of claim 1, wherein the first suction tube has an inner tube volume extending over a full length of the first suction tube, and the internal volume between the first shell and the second shell is greater than the inner tube volume.

12. The breast pump system of claim 1, wherein the charging chamber has a charging chamber volume that is less than the internal volume between the first shell and the second shell.

13. A breast pump system that uses air for assisting a lactating woman in collecting milk expressed by a breast of the lactating woman, the breast pump system comprising:
a vacuum pump defining an air chamber, the vacuum pump being operable cyclically between a negative pressure state and a positive pressure state, the air in the air chamber being at a first negative pressure when the vacuum pump is in the negative pressure state, the air in the air chamber being at a first positive pressure when the vacuum pump is in the positive pressure state;
a milk collection device being configured to fittingly receive the breast of the lactating woman, the milk collection device defining a charging chamber that is in fluid communication with the breast when the breast fittingly engages the milk collection device;
a first suction tube being connected to the milk collection device and being connected in fluid communication with the charging chamber;
a fluid isolator coupling the first suction tube to the vacuum pump, the fluid isolator comprising a shell and a diaphragm; the shell defining an internal volume, a first port and a second port; the internal volume having a first chamber and a second chamber that are separated by the diaphragm; the first port connecting the first chamber in fluid communication with the first suction tube; the second port connecting the second chamber in fluid communication with the air chamber of the vacuum pump; and
a central portion of the diaphragm being pliable and limp within the internal volume of the fluid isolator when both the first chamber and the second chamber contain air at atmospheric air pressure, and the central portion of the diaphragm having selectively a relaxed position that is neither biased toward the first port nor biased toward the second port.

14. The breast pump system of claim 13, wherein the first suction tube has an inner tube volume extending over a full length of the first suction tube, and the internal volume of the shell is greater than the inner tube volume.

15. The breast pump system of claim 13, wherein the charging chamber has a charging chamber volume that is less than the internal volume of the shell.

16. A breast pump system that uses air for assisting a lactating woman in collecting milk expressed by a breast of the lactating woman, the breast pump system comprising:
a vacuum pump defining an air chamber, the vacuum pump being operable cyclically between a negative pressure state and a positive pressure state, the air in the air chamber being at a first negative pressure when the vacuum pump is in the negative pressure state, the air in the air chamber being at a first positive pressure when the vacuum pump is in the positive pressure state;
a milk collection device being configured to fittingly receive the breast of the lactating woman, the milk collection device defining a charging chamber that is in fluid communication with the breast when the breast fittingly engages the milk collection device, the charging chamber having a charging chamber volume;
a first suction tube being connected to the milk collection device and being connected in fluid communication with the charging chamber, the first suction tube having an inner tube volume extending over a full length of the first suction tube; and
a fluid isolator coupling the first suction tube to the vacuum pump, the fluid isolator comprising a shell and a diaphragm; the shell defining an internal volume, a first port and a second port; the diaphragm being moveable between the first port and the second port; the internal volume having a first chamber and a second chamber that are separated by the diaphragm; the first port connecting the first chamber in fluid communication with the first suction tube; the second port connecting the second chamber in fluid communication with the air chamber of the vacuum pump; the internal volume of the shell being greater than the inner tube volume; and the internal volume of the shell being greater than the charging chamber volume.

17. The breast pump system of claim 16, wherein the shell comprises a see-through material.

18. The breast pump system of claim 16, wherein a central portion of the diaphragm is pliable and limp within the internal volume of the shell when both the first chamber and the second chamber contain air at atmospheric air pressure.

19. The breast pump system of claim 16, wherein a central portion of the diaphragm has a relaxed position halfway between the first port and the second port.

20. The breast pump system of claim 16, wherein diaphragm connects to the shell at a joint that circumscribes a cross-sectional area of the internal volume, the diaphragm has a surface area facing the first port, and the surface area is greater than the cross-sectional area of the internal volume.
